(19) United States
(12) Patent Application Publication
(10) Pub. No.: US 2025/0367448 A1
(43) Pub. Date: Dec. 4, 2025

(54) SYSTEMS AND METHODS FOR EVOKED SIGNAL SENSING USING ADJUSTABLE DC OFFSET COMPENSATION

(71) Applicant: Boston Scientific Neuromodulation Corporation, Valencia, CA (US)

(72) Inventors: Soroush Niketeghad, Stevenson Ranch, CA (US); Nathan Maas, Simi Valley, CA (US); Kiran K. Gururaj, Valencia, CA (US)

(21) Appl. No.: 19/224,248

(22) Filed: May 30, 2025

Related U.S. Application Data
(60) Provisional application No. 63/655,935, filed on Jun. 4, 2024.

(51) Int. Cl. A61N 1/36

(52) U.S. Cl. CPC A61N 1/36125; A61N 1/3603; A61N 1/36132

(57) ABSTRACT

A stimulation system includes a stimulation generation system to provide stimulation; an evoked signal sensing system to receive an evoked signal from the tissue in response to the stimulation; and a processing system. The evoked signal sensing system includes a sense amplifier circuit and a DC offset compensation circuit configured to provide DC offset compensation at an input of the sense amplified circuit. The processing system is configured to, during the providing of the stimulation, direct the DC offset compensation circuit to provide, DC offset compensation at a first magnitude; and, after the providing of the stimulation and during a sensing window for the evoked signal, either a) direct the DC offset compensation circuit to provide DC offset compensation at a second magnitude that is no more than one-third of the first magnitude or b) direct the DC offset compensation circuit to provide no DC offset compensation.

### CROSS-REFERENCE TO RELATED APPLICATIONS

This application claims the benefit under 35 U.S.C. § 119(e) of U.S. Provisional Patent Application Ser. No. 63/655,935, filed Jun. 4, 2024, which is incorporated herein by reference.

### FIELD

The present disclosure is directed to the area of evoked signal sensing systems and methods of making and using the systems. The present disclosure is also directed to stimulation systems that include evoked signal sensing and methods of making and using the stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Deep brain stimulation can be used to treat a variety of diseases and disorders.

Stimulators have been developed to provide therapy for a variety of treatments. A stimulator can include a control module (with a pulse generator) and one or more stimulator electrodes. The one or more stimulator electrodes can be disposed along one or more leads, or along the control module, or both. The stimulator electrodes are in contact with or near the nerves, muscles, or other tissue to be stimulated. The pulse generator in the control module generates electrical pulses that are delivered by the electrodes to body tissue.

An evoked signal is electrical activity of the tissue in response to stimulation. It can be desirable to measure or otherwise sense evoked signals. For example, the evoked signals can be measured or sensed from a region of the body such as the cerebral cortex, brain stem, spinal cord, a peripheral nerve, a muscle, or the like. For instance, an evoked potential in response to neurostimulation provided by an implantable neurostimulation device can be measured or sensed.

### BRIEF SUMMARY

One aspect is a stimulation system that includes a stimulation generation system configured to generate stimulation signals to provide stimulation to tissue of a patient; an evoked signal sensing system configured to receive an evoked signal from the tissue in response to the stimulation, wherein the evoked signal sensing system includes a sense amplifier circuit configured to receive, at a node, a sense amplification input signal that is based, at least in part, on the received evoked signal, and provide a sense amplification output signal by amplifying the sense amplification input signal, and a DC offset compensation circuit configured to provide DC offset compensation at the node; and a processing system configured to, during the providing of the stimulation, directing the DC offset compensation circuit to provide, at the node, the DC offset compensation at a first magnitude; and, after the providing of the stimulation and during a sensing window for the evoked signal, either a) directing the DC offset compensation circuit to provide, at the node, the DC offset compensation at a second magnitude that is no more than one-third of the first magnitude or b) directing the DC offset compensation circuit to provide, at the node, no DC offset compensation.

In at least some aspects, during the sensing window, the processing system is configured to direct the DC offset compensation circuit to provide, at the node, the DC offset compensation at the second magnitude that is no more than one-tenth of the first magnitude. In at least some aspects, during the sensing window, the processing system is configured to direct the DC offset compensation circuit to provide, at the node, no DC offset compensation.

In at least some aspects, the sense amplifier circuit includes at least one differential amplifier. In at least some aspects, the stimulation system further includes at least one DC-blocking capacitor coupled, or coupleable, to the node. In at least some aspects, the stimulation system further includes at least one electrical stimulation lead, the at least one stimulation lead including a plurality of electrodes including at least one first electrode and at least one second electrode, wherein the processing system is configured to direct the stimulation generation system to provide the stimulation through the at least first electrode. In at least some aspects, the sense amplifier circuit is coupled, or coupleable, to the at least one second electrode for receiving the evoked signal from the tissue. In at least some aspects, when the sense amplifier circuit is coupled to a one of the at least one second electrode, at least one of the at least one DC-blocking capacitor is coupled between the one of the at least one second electrode and the node.

In at least some aspects, the stimulation system is configured for at least one of deep brain stimulation, spinal cord stimulation, peripheral nerve stimulation, dorsal root ganglion stimulation, or sacral nerve stimulation. In at least some aspects, the stimulation system further includes an implantable pulse generator, wherein stimulation generation system and the sense amplifier circuit are part of the implantable pulse generator. In at least some aspects, the stimulation system further includes an external stimulator, wherein stimulation generation system and the sense amplifier circuit are part of the external stimulator.

A further aspect is an electrical stimulation system that includes at least one electrical stimulation lead including a plurality of electrodes; and a stimulator coupled or coupleable to the at least one electrical stimulation lead. The stimulator includes stimulation generation circuitry configured to provide stimulation to tissue of a patient via one or more of the electrodes; an evoked signal sensing arrangement configured to receive, from at least one of the electrodes, an evoked signal from the tissue in response to the stimulation; and a processor. The evoked signal sensing arrangement includes a sense amplifier circuit configured to receive, at a node, a sense amplification input signal that is based, at least in part, on the evoked signal received from the at least one of the electrodes, and provide a sense amplification output signal by amplifying the sense amplification input signal, and a DC offset compensation circuit configured to provide DC offset compensation at the node. The processor is configured to, during the providing of the stimulation, directing the DC offset compensation circuit to provide, at the node, the DC offset compensation at a first magnitude; and, after the providing of the stimulation and during a sensing window for the evoked signal, either a) directing the DC offset compensation circuit to provide, at the node, the DC offset compensation at a second magnitude that is no more than one-third of the first magnitude or b) directing the DC offset compensation circuit to provide, at the node, no DC offset compensation.

In at least some aspects, the stimulator is an implantable pulse generator. In at least some aspects, the stimulator is an external trial stimulator. In at least some aspects, the stimulation system further includes at least one DC-blocking capacitor coupled, or coupleable, to the node, wherein at least one of the at least one DC-blocking capacitor is coupled between a one of the electrodes and the node. In at least some aspects, when the sense amplifier circuit is coupled to the one of the electrodes, at least one of the at least one DC-blocking capacitor is coupled between the one of the electrodes and the node.

Another aspect is a method for sensing an evoked signal using any of the stimulation systems described above. The method includes providing stimulation to the patient using the stimulation generation system; during the providing of the stimulation, providing, at the node of the sense amplifier circuit, the DC offset compensation at a first magnitude; halting the stimulation; and after the halting of the stimulation and during a sensing window for the evoked signal, either a) providing, at the node, the DC offset compensation at a second magnitude that is no more than one-third of the first magnitude or b) providing, at the node, no DC offset compensation.

In at least some aspects, after the halting of the stimulation and during the sensing window for the evoked signal, the DC offset compensation provided, at the node, is at the second magnitude that is no more than one-tenth of the first magnitude. In at least some aspects, after the halting of the stimulation and during the sensing window for the evoked signal, no DC offset compensation is provided at the node. In at least some aspects, the stimulation generation system and the sense amplifier circuit are implanted in the patient.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, in which:

FIG. 1 is a schematic view of an embodiment of an electrical stimulation system;

FIG. 2 is a block diagram of an embodiment of a system for sensing an evoked signal;

FIG. 3 is a graph of DC offset compensation current versus time for stimulation and a subsequent sense window;

FIG. 4 is a block diagram of an embodiment of the system of FIG. 1;

FIG. 5 is a flowchart of one embodiment of a method of sensing an evoked signal; and

FIG. 6 is a schematic overview of an embodiment of components of a stimulation and sensing system, including an electronic subassembly disposed within a control module.

### DETAILED DESCRIPTION

The present disclosure is directed to the area of evoked signal sensing systems and methods of making and using the systems. The present disclosure is also directed to stimulation systems that include evoked signal sensing and methods of making and using the stimulation systems.

An evoked signal is electrical activity of the tissue in response to stimulation. It can be desirable to measure or otherwise sense evoked signals. For example, evoked signals can be measured or otherwise sensed after stimulation of a region of the body such as the cerebral cortex, brain stem, spinal cord, a peripheral nerve, a muscle, or the like. For instance, an evoked signal in response to neurostimulation provided by an implantable neurostimulation device can be measured or otherwise sensed.

Examples of electrical stimulation systems that can be modified as described herein to include sensing capabilities are found in, for example, U.S. Pat. Nos. 6,181,969; 6,295,944; 6,391,985; 6,516,227; 6,609,029; 6,609,032; 6,741,892; 7,244,150; 7,450,997; 7,672,734; 7,761,165; 7,783,359; 7,792,590; 7,809,446; 7,949,395; 7,974,706; 8,831,742; 8,688,235; 8,175,710; 8,224,450; 8,271,094; 8,295,944; 8,364,278; and 8,391,985; U.S. Patent Application Publications Nos. 2007/0150036; 2009/0187222; 2009/0276021; 2010/0076535; 2010/0268298; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; 2011/0005069; 2010/0268298; 2011/0130817; 2011/0130818; 2011/0078900; 2011/0238129; 2011/0313500; 2012/0016378; 2012/0046710; 2012/0165911; 2012/0197375; 2012/0203316; 2012/0203320; and 2012/0203321, all of which are incorporated by reference in their entireties. Examples of devices for measuring or otherwise sensing an evoked potential (e.g., a neural response) are found in, for example, U.S. Pat. Nos. 7,385,443; 11,040,202; 11,633,138; and U.S. Patent Application Public Nos. 2022/0007808, 2022/0007980, 2023/0173273, 2024/0058611, all of which are incorporated herein by reference.

Electrical stimulation systems often include at least one lead with one or more electrodes disposed on a distal portion of the lead and one or more terminals disposed on one or more proximal portions of the lead. Leads include, for example, percutaneous leads, paddle leads, cuff leads, or any other arrangement of electrodes on a lead.

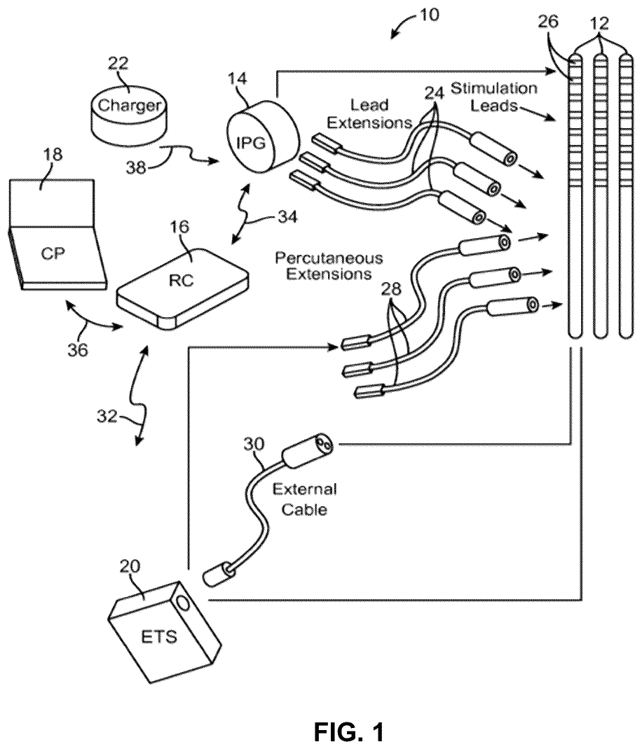

Turning to FIG. 1, one embodiment of an electrical stimulation system 10 includes one or more stimulation leads 12 and an implantable pulse generator (IPG) 14. The stimulation system 10 can also include one or more of an external remote control (RC) 16, a clinician's programmer (CP) 18, an external trial stimulator (ETS) 20, or an external charger 22. The IPG and ETS are examples of control modules for the electrical stimulation system. The ETS 20 is a type of external stimulator and will be used herein as an example, but it will be understood that any other external stimulator can be used in the place of the ETS 20.

The IPG 14 is physically connected, in at least some embodiments, via one or more lead extensions 24, to the stimulation lead(s) 12. In at least some embodiments, each lead carries multiple electrodes 26 arranged in an array. In at least some embodiments, the IPG 14 includes pulse generation circuitry that delivers electrical stimulation energy in the form of, for example, a pulsed electrical waveform (i.e., a temporal series of electrical pulses) to the electrode array 26 in accordance with a set of stimulation parameters. The IPG 14 can be implanted into a patient's body, for example, below the patient's clavicle area or within the patient's buttocks or abdominal cavity or at any other suitable site.

The IPG 14 can have multiple stimulation channels which may be independently programmable to control the magnitude of the current stimulus from each channel. In at least some embodiments, the IPG 14 can have any suitable number of stimulation channels including, but not limited to, 4, 6, 8, 12, 16, 32, or more stimulation channels. In various embodiments, the IPG 14 can have one, two, three, four, or more connector ports, for receiving the terminals of the leads and/or lead extensions.

The ETS 20 may also be physically connected, in at least some embodiments, via the percutaneous lead extensions 28 and the external cable 30, to the stimulation leads 12. The ETS 20, which may have similar pulse generation circuitry as the IPG 14, also delivers electrical stimulation energy in the form of, for example, a pulsed electrical waveform to the electrode array 26 in accordance with a set of stimulation parameters. One difference between the ETS 20 and the IPG 14 is that, in at least some embodiments, the ETS 20 is a non-implantable device that is used on a trial basis after the neurostimulation leads 12 have been implanted and prior to implantation of the IPG 14, to test the responsiveness of the stimulation that is to be provided. Any functions described herein with respect to the IPG 14 can likewise be performed with respect to the ETS 20 in at least some embodiments.

The RC 16 may be used to telemetrically communicate with or control the IPG 14 or ETS 20 via a uni- or bi-directional wireless communications link 32. Once the IPG 14 and neurostimulation leads 12 are implanted, the RC 16 may be used to telemetrically communicate with or control the IPG 14 via a uni- or bi-directional communications link 34. In at least some embodiments, such communication or control allows the IPG 14 to be turned on or off and to be programmed with different stimulation parameter sets.

The IPG 14 may also be operated to modify the programmed stimulation parameters to actively control the characteristics of the electrical stimulation energy output by the IPG 14. In at least some embodiments, the CP 18 allows a user, such as a clinician, the ability to program stimulation parameters for the IPG 14 and ETS 20 in the operating room and in follow-up sessions. Alternately, or additionally, stimulation parameters can be programed via wireless communications (e.g., Bluetooth) between the RC 16 (or external device such as a hand-held electronic device) and the IPG 14.

In at least some embodiments, the CP 18 may perform this function by indirectly communicating with the IPG 14 or ETS 20, through the RC 16, via a wireless communications link 36. Alternatively, in at least some embodiments, the CP 18 may directly communicate with the IPG 14 or ETS 20 via a wireless communications link (not shown). In at least some embodiments, the stimulation parameters provided by the CP 18 are also used to program the RC 16, so that the stimulation parameters can be subsequently modified by operation of the RC 16 in a stand-alone mode (i.e., without the assistance of the CP 18).

Stimulation provided by IPG 14 is typically provided by pulses, each of which may include a number of phases. Stimulation parameters typically include amplitude (current I, although a voltage amplitude V can also be used); frequency (F); pulse width (PW); the electrodes 26 selected to provide the stimulation; and the polarity of such selected electrodes, i.e., whether they act as anodes that source current to the tissue or cathodes that sink current from the tissue. In at least some embodiments, these and possibly other stimulation parameters taken together comprise a stimulation program that the stimulation circuitry 229 in the IPG 14 can execute to provide therapeutic stimulation to a patient.

For purposes of brevity, the details of the RC 16, CP 18, ETS 20, and external charger 22 will not be further described herein. Details of exemplary embodiments of these devices are disclosed in U.S. Pat. No. 6,895,280, which is expressly incorporated herein by reference. Other examples of electrical stimulation systems can be found at U.S. Pat. Nos. 6,181,969; 6,516,227; 6,609,029; 6,609,032; 6,741,892; 7,949,395; 7,244,150; 7,672,734; and 7,761,165; 7,974,706; 8,175,710; 8,224,450; and 8,364,278; and U.S. Patent Application Publication No. 2007/0150036, as well as the other references cited above, all of which are incorporated herein by reference.

In at least some embodiments, a percutaneous lead for electrical stimulation (for example, deep brain, spinal cord, or peripheral nerve stimulation) includes stimulation electrodes that can be ring electrodes, segmented electrodes that extend only partially around the circumference of the electrical stimulation lead, or any other type of electrode, or any combination thereof. The segmented electrodes can be provided in sets of electrodes, with each set having electrodes circumferentially distributed about the lead at a particular longitudinal position. A set of segmented electrodes can include any suitable number of electrodes including, for example, two, three, four, or more electrodes. For illustrative purposes, the systems and leads are described herein relative to use for deep brain stimulation, but it will be understood that any of the leads can be used for applications other than deep brain stimulation, including spinal cord stimulation, peripheral nerve stimulation, dorsal root ganglion stimulation, sacral nerve stimulation, or stimulation of other nerves, muscles, and tissues. Also, in at least some embodiments, the stimulation generation system 129 is lead-less and accomplishes stimulation without the use of leads.

Electrodes of the lead(s) (or electrodes of a sensor or other device) can be used to sense the evoked signal, which may include sensing local electrical characteristics of the environment around the lead(s) and electrodes during and between electrical pulses or waveforms (which can be, for example, therapeutic stimulation pulses or waveforms, sub-perception pulses or waveforms, sensing pulses or waveforms, or other electrical pulses or waveforms).

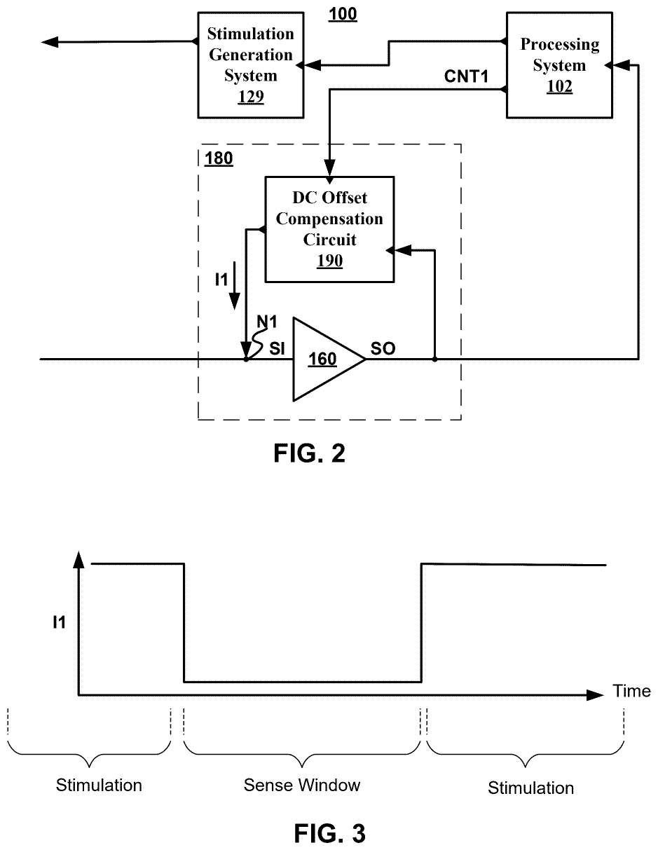

FIG. 2 illustrates one embodiment of a stimulation and sensing arrangement 100 that includes a stimulation generation system 129, an evoked signal sensing system 180, and a processing system 102. The evoked signal sensing system 180 includes a sense amplifier circuit 160 and a DC offset compensation circuit 190.

Referring to FIGS. 1 and 2, the stimulation generation system 129 can be part of, for example, the IPG 14 or ETS 20. The evoked signal sensing system 180 can also be part of the IPG 14 or ETS 20 or can be a separate device. The processing system 102 can be part of the IPG 14, ETS 20, CP 18, RC 16, or any other suitable device or can be distributed between two or more of the IPG 14, ETS 20, CP 18, RC 16, or any other suitable device. Any other suitable distribution of the components of the stimulation and sensing arrangement 100 can be used.

In at least some embodiments, the stimulation and sensing arrangement 100 is arranged as follows. The stimulation generation system 129 has at least a first input and a first output. The sense amplifier circuit 160 has at least a first input and a first output. The first input of the sense amplifier circuit 160 is coupled to a node N1. The DC offset compensation circuit 190 has at least a first input and a first output. The first output of the DC offset compensation circuit 190 is coupled to the node N1. The processing system 102 has at least a first input, a first output, and a second output. The first input of the processing system 102 is coupled to the first output of the sense amplifier circuit 160, the first output of the processing system 102 is coupled to the first input of the stimulation generation system 129, and the second output of the processing system 102 is coupled to the first input of the DC offset compensation circuit 190. In at least some embodiments, the first output of the sense amplifier circuit 160 can be coupled as an optional second input into the DC offset compensation circuit 190.

The processing system 102 may include one or more microcontrollers (MCUs), microprocessors, field-programmable gate arrays (FPGAs), digital-signal processors (DSPs), application-specific integrated circuits (ASICs), or other suitable devices. In at least some embodiments, at least a portion of the processing system 102 is at least a portion of a control module, such as IPG 14 or ETS 20.

The stimulation generation system 129 is arranged to provide stimulation (e.g., electrical stimulation) to tissue of a patient based on control provided by the processing system 102. The tissue can be, for example, the spinal cord, a dorsal root ganglia, a peripheral nerve, one or more brain structures, or the like. In at least some embodiments, at least a portion of the stimulation generation system 129 is implanted in the patient's body and provides stimulation via one or more implanted electrodes coupled to the stimulation generation system 129. Various embodiments of the stimulation generation system 129 provide deep brain stimulation, spinal cord stimulation, peripheral nerval stimulation, dorsal root ganglion stimulation, sacral nerve stimulation, or stimulation of other nerves, neural tissue, or other tissues. In at least some embodiments, the electrical stimulation provided by the stimulation generation system 129 may have any suitable beneficial therapeutic effect.

The tissue provides an evoked signal in response to the stimulation provided by the stimulation generation system 129. The evoked signal sensing system 180 is arranged to receive and sense the evoked signal. The evoked signal may include a response signal, such as a neural response signal, or can include any electrophysiological signal that is generated or altered in response to the stimulation. The evoked signal can be an evoked potential, evoked compound action potential (ECAP), a tissue response, evoked resonant neural activity (ERNA), local field potential (LFP), ESG (electrospinogram), EEG (electroencephalogram), ECG (electrocardiogram), ECoG (electrocorticogram), or EMG (electromyogram) signal or the like or any combination thereof. Examples of systems for sensing evoked signals can be found at, for example, U.S. Pat. No. 11,040,202 and U.S. Patent Application Publications Nos. 2020/0251899; 2021/0236829; 2020/0305744; 2023/0173273; 2023/0248978; and 2024/0058611, all of which are incorporated herein by reference in their entireties. It will also be understood that the evoked signal sensing system 180 can be used to measure or sense other electrophysiological signals.

In at least some embodiments, the sensing of the evoked signal is performed via components, at least some of which are implanted in the body of the patient. In at least some embodiments, the sensing of the evoked signal is performed using one or more components that are external to the patient.

The evoked signal sensing system 180 can sense an evoked signal produced by the tissue, which has received electrical stimulation by stimulation generation system 129. In at least some embodiments, the processing system 102 records, measures, observes, or outputs the sensed evoked signal. In at least some embodiments, instead of or in addition to recording the sense evoked signal, the processing system 102 analyzes the sensed evoked signal to make various determinations, such as, for example, determining the efficacy of the stimulation in producing at least one therapeutic effect.

Evoked signals can arise, for example, from those neurons directly activated by electrical stimulation from the stimulation generation system 129. Evoked signals may also reflect the propagation of neural activity across a neural network and may reflect both local and network level activation. In at least some embodiments, when a neural fiber is recruited by electrical stimulation caused by the stimulation generation system 129, the neural fiber, in response, will issue an action potential—that is, the neural fiber will “fire.” In at least some embodiments, should recruitment from electrical stimulation result in the neural fiber's activation, the neural fiber may depolarize, repolarize, and hyperpolarize before coming to rest again. If electrical stimulation continues, the neural fiber may fire again at some later time.

The sense amplifier circuit 160 is arranged to receive a sense amplification input signal SI at node N1. The sense amplification input signal SI is based, at least in part, on the evoked signal. The sense amplifier circuit 160 is further arranged to provide a sense amplification output signal SO by amplifying the sense amplification input signal SI. As discussed in greater detail below, in at least some embodiments, the sense amplification output signal SO is received and processed by processing system 102. In at least some embodiments, the processing system 102 may determine whether to perform further actions based on the sensed evoked signal. For instance, in at least some embodiments, based on the sensed evoked signal, the processing system 102 causes the stimulation provided by the stimulation generation system 129 to be altered or adjusted to improve or alter treatment provided to the patient. In at least some embodiments, rather than, or in addition to, determining whether to alter the treatment, the processing system 102 records and stores the sensed evoked signal or outputs the sensed evoked signal to a user or another system.

The DC offset compensation circuit 190 is arranged to provide current I1 to node N1. Node N1 has, or is associated with, a capacitance. The DC offset compensation circuit 190 is arranged such that the magnitude of current I1 is adjustable based on a control signal CNT1, which directs the DC offset compensation circuit 190 to provide DC offset compensation at node N1. As discussed in greater detail below, in at least some embodiments, the DC offset compensation provided by the DC offset compensation circuit 190 can be used to prevent, or reduce the likelihood of, saturation of the sense amplifier circuit 160. Without the DC offset compensation, the sense amplifier circuit 160 may be saturated so that little or no information related to the evoked signal can be sensed or recorded.

Node N1 has, or is coupled to, a capacitance, such as the capacitance of a DC-blocking capacitor or other suitable capacitance. The voltage change at node N1 does not happen instantaneously. That is, the voltage at node N1 does not increase to the desired offset level instantaneously, nor does the voltage offset change instantaneously when current is no longer being injected to node N1. Rather, the speed at which the DC offset compensation occurs depends on the amount of capacitance at node N1 and on the magnitude of current I1.

In at least some embodiments, the processing system 102 is arranged to provide the first control signal CNT1 to direct the DC offset compensation circuit 190 to provide current I1. Conventionally, the DC offset compensation circuit 190 provides a current I1 with the same, or nearly the same, magnitude before, during, and after stimulation. This DC offset compensation current I1 prevents, or reduces the likelihood of, saturation of the sense amplifier circuit 160 arising from the relatively strong stimulation signal produced by the stimulation generation system 129.

In contrast to this conventional application of a DC offset compensation current, it has been found that substantially reducing or halting the current I1 during a sensing window after stimulation can facilitate sensing of evoked signals during that sensing window. For example, when the stimulation generation system 129 is providing electrical stimulation to the tissue, the processing system 102 directs the DC offset compensation circuit 190 to provide current I1 with a first magnitude at node N1. After providing the stimulation and during a sensing window (in which no stimulation is provided and during which the evoked signal is expected to be received), in at least some embodiments, the processing system 102 directs the DC offset compensation circuit 190 to provide current I1 with a second magnitude at node N1. The second magnitude is no more than one third (33%), one-fourth (25%), one-tenth (10%), or one-twentieth (5%) of the first magnitude. In other embodiments, after providing the stimulation and during the sensing window, the processing system 102 directs the DC offset compensation circuit 190 to provide no current I1 at node N1 (i.e., I1=0 Amps). In these embodiments, there is no DC offset compensation provided by the DC offset compensation circuit 190.

In any of these embodiments, after the sensing window or when the stimulation is to be provided again, the processing system 102 directs the DC offset compensation circuit 190 to return to providing current I1 with the first magnitude at node N1.

FIG. 3 is a graph of current I1 versus time for one embodiment. As an example, in one embodiment, the DC offset compensation circuit 190 provides a current I1 of several hundred nA when the stimulation generation system 129 is providing electrical stimulation to the tissue of the patient. After delivery of the stimulation and during the sensing window, the current I1 is reduced to less than 100 or 50 nA, as illustrated in FIG. 3. In another example, the DC offset compensation circuit 190 provides no current I1 during the sensing window. After the sensing window or when the stimulation is to be provided again, the processing system 102 then directs the DC offset compensation circuit 190 to return current I1 to the first magnitude.

In at least some embodiments, by removing or substantially reducing the magnitude of current I1 during the sensing window, DC offset compensation is still achieved, so that the sense amplifier circuit 160 does not become saturated by the stimulation signal from the stimulation generation system 129. In at least some embodiments, the sensing window occurs between stimulation periods, as illustrated in FIG. 3, and has a duration of, for example, no more than 100, 50, 25, 10, 6, or 5 milliseconds.

In at least some embodiments, by removing or substantially reducing the magnitude of current I1 during the sensing window, the signal-to-noise ratio (SNR), which decreases with increased magnitude of I1, of the amplification output signal SO remains high. The higher the SNR achieved by using this dynamic DC offset compensation scheme, the larger a population of patients may obtain usable information from the evoked signal by the processing system 102. By managing the DC offset compensation in this dynamic manner, a useful trade-off between saturation and SNR of the amplification output signal SO can be achieved.

Although the processing system 102 is shown as one component in FIG. 2, in at least some embodiments, the processing system 102 is a system that includes multiple separate systems. For instance, in at least some embodiments, the processing system 102 includes one system for receiving and processing the sense amplification output signal SO and another separate system for controlling the stimulation generation system 129. These systems may be separate from each other or combined together. As discussed above, in various embodiments of the stimulation and sensing arrangement 100, the sensing of the evoked signal may be via an implanted device, may be done externally, or both.

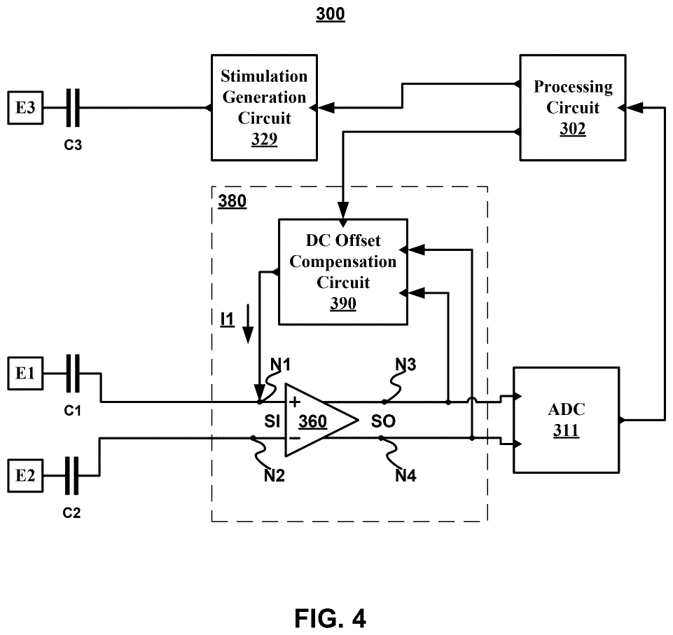

FIG. 4 is a block diagram of one embodiment of a system 300. The system 300 is an embodiment of the stimulation and sensing arrangement 100 of FIG. 2. The system 300 includes a processing circuit 302, a stimulation generation circuit 329, electrodes E1-E3 (for example, electrodes 26 of one or more stimulation leads 12 (FIG. 1)), DC-blocking capacitors C1-C3, an evoked signal sensing system 380, and an analog-to-digital converter (ADC) 311. The evoked signal sensing system 380 includes a sense amplifier circuit 360 and a DC offset compensation circuit 390. Although a particular stimulation and sense configuration is illustrated in FIG. 4 and discussed herein by way of example, a variety of different suitable stimulation/sense configurations may be used in various embodiments of the system 300. Among other things, in various embodiments, the system 300 can be used for bipolar or multipolar stimulation as well as monopolar stimulation, can be used for bipolar, multipolar, or monopolar sensing, and can be used for stimulation or sensing of a variety of different suitable portions of a patient's body.

In at least some embodiments, the system 300 is arranged as follows. The stimulation generation circuit 329 has at least a first input and a first output. The sense amplifier circuit 360 has at least a first input, a second input, a first output, and a second output. The first input of the sense amplifier circuit 360 is coupled to node N1, the second input of the sense amplifier circuit 360 is coupled to node N2, the first output of the sense amplifier circuit 360 is coupled to node N3, and the second output of the sense amplifier circuit 360 is coupled to node N4. The DC offset compensation circuit 390 has at least a first input and an output. The first input of the DC offset compensation circuit 390 is coupled to the processing circuit 302 and the first output of the DC offset compensation circuit 390 is coupled to node N1. In at least some embodiments, an optional second input of the DC offset compensation circuit 390 is coupled to node N3. In at least some embodiments, an optional third input of the DC offset compensation circuit 390 is coupled to node N4. The ADC 311 has at least a first input, a second input, and a first output. The first input of the ADC 311 is coupled to node N3, and the second input of the ADC 311 is coupled to node N4. The processing circuit 302 has at least a first input, a first output, and a second output. The first input of the processing circuit 302 is coupled to the first output of the ADC 311, the first output of the processing circuit 302 is coupled to the first input of the stimulation generation circuit 329, and the second output of the processing circuit 302 is coupled to the first input of the DC offset compensation circuit 390. DC-blocking capacitor C1 is coupled between node N1 and electrode E1. DC-blocking capacitor C2 is coupled between node N2 and electrode E2. DC-blocking capacitor C3 is coupled between electrode E3 and the first output of the stimulation generation circuit 329.

The stimulation generation circuit 329 is an embodiment of the stimulation generation system 129 of FIG. 2. As discussed above, the stimulation generation circuit 329 is arranged to provide stimulation (e.g., electrical stimulation) to tissue of a patient based on control from the processing circuit 302. The processing circuit 302 is an embodiment of the processing system 102 of FIG. 2.

In at least some embodiments, the electrodes (e.g., E1-E3) include electrodes used for stimulation and electrodes used for sensing. In at least some embodiments, each electrode can be used for stimulation or for sensing. For instance, in the embodiment illustrated in FIG. 4, electrodes E1 and E2 are used for sensing and electrode E3 is used for stimulation. While FIG. 4 illustrates three electrodes E1-E3, other embodiments of system 300 may include more or fewer electrodes. Stimulation and sensing can be monopolar (for example, using the case of the IPG 14 as a return electrode) or multipolar (for example, bipolar). The references cited above illustrate monopolar and multipolar stimulation. Similarly, the electrodes can be used for monopolar or multipolar sensing.

Although not shown in FIG. 4, in at least some embodiments, the system 300 further includes a multiplexer to select from among multiple electrodes, e.g., to select sensing electrodes to be used to sense the evoked signal and to select electrodes to be used for stimulation. In at least some embodiments, when multiple electrodes can be selected from, the electrodes can be determined automatically by the processing circuit 302 or selected by a user via the processing circuit 302.

In at least some embodiments, the stimulation generation circuit 329 is arranged to provide electrical stimulation to tissue of a patient via one or more electrodes, such as electrode E3, under the control of the processing circuit 302. In at least some embodiments, the sense amplifier circuit 360 is implantable in the patient. In other embodiments, the sense amplifier circuit 360 is external to the patient. Other components of the system 300 may be external to the patient or implanted in the patient. For instance, as discussed above, at least some embodiments employ an IPG that is implanted in a patient, and at least some embodiments employ an ETS that is external to the patient. In at least some embodiments, as discussed above, the ETS is an external device that is used on a trial basis after leads have been implanted and prior to implantation of the IPG, to test the responsiveness of the stimulation that is to be provided. In embodiments that include implantable components, the implantable components are biocompatible.

In at least some embodiments, the current paths to the electrodes E1, E2, and E3 include DC-blocking capacitors C1, C2, and C3, respectively. In at least some embodiments, when there are more than three electrodes, the system 300 also has a corresponding DC-blocking capacitor for each of the additional electrodes. In at least some embodiments, the DC-blocking capacitors C1-C3 prevent the inadvertent supply of DC current to the electrodes E1-E3 and from there to a patient's tissue. In at least some embodiments, at least some of the electrodes E1-E3 may be positioned on one or more leads, such as the simulation leads 12 of FIG. 1.

The system 300 is arranged so that one or more of the electrodes, for example, the electrodes E1 and E2, receive evoked signals in response to the electrical stimulation provided by the stimulation generation circuit 329. The evoked signal are typically relatively small-amplitude signals, on the order of microvolts or millivolts, which can make sensing difficult, particularly in view of the much larger stimulation signal. The sense amplifier circuit 360 is arranged to sense and amplify the evoked signal.

The sense amplifier circuit 360 includes one or more sense amplifiers. In at least some embodiments, the sense amplifier circuit 360 contains exactly one sense amplifier. In at least some embodiments, the sense amplifier circuit 360 includes two or more sense amplifier coupled in series, so that the output of one sense amplifier in the sense amplifier circuit 360 is coupled to the input of another sense amplifier in the sense amplifier circuit 360, in order to add further gain. In at least some embodiments, the sense amplifier circuit 360 includes two or more sense amplifiers in which one of the sense amplifiers is selected for use depending on the nature of sense amplifier input signal SI. In at least some embodiments, the sense amplifier circuit 360 contains two or more sense amplifiers for sensing using different electrodes.

In at least some embodiments, one or more sense amplifiers of the sense amplifier circuit 360 is a differential amplifier. In at least some embodiments, one or more of the sense amplifiers of the sense amplifier circuit 360 is a bioamplifier that can safely operate while implanted in the body of a patient. The sense amplifier circuit 360 may also include other circuits such as one or more switches, a filter circuit at the output of the sense amplifier (such as a low-pass filter, a high-pass filter, or band-pass filter), or any other suitable circuit.

The sense amplifier circuit 360 receives sense amplifier input signal SI and outputs sense amplifier circuit output signal SO. In the embodiment illustrated in FIG. 4, signals SI and SO are both differential signals, with differential input signal SI received by the sense amplifier circuit 360 at nodes N1 and N2 and differential output signal SO provided at nodes N3 and N4 by the sense amplifier circuit 360. However, in other embodiments, one or both of signals SI and SO are single-ended signals.

In at least some embodiments, the ADC 311 is arranged to convert the sense amplification output signal SO into a digital signal and to provide the digital signal output by ADC 311 to the processing circuit 302. In other embodiments, the ADC 311 is excluded from the system 300, or is included as part of the processing circuit 302, and the processing circuit 302 receives sense amplification output signal SO directly. In at least some embodiments, in response to the received digital signal output from the ADC 311, the processing circuit 302 processes the received digital signal.

The processing of the digital signal by the processing circuit 302 may include, for example, recording information associated with the digital signal or determining whether to perform further actions based on the received digital signal. For instance, in at least some embodiments, based on the received digital signal, which is based on the sensed evoked signal, the processing circuit 302 may cause the electrical stimulation provided by the stimulation generation circuit 329 to be altered or adjusted in such a way that a better treatment is provided to the patient. In at least some embodiments, the processing circuit 302 is arranged to provide information to the user regarding the evoked signal, to determine whether a warning should be provided to a user based on the evoked signal, or the like. In at least some embodiments, information associated with the evoked signal is recorded and stored or output to the user or another system.

There may be artifacts in the sensed signals arising from the electrical stimulation of the tissue. The artifacts from the electrical stimulation of the first target can cause the sense amplifier circuit 360 to saturate so that none of the information from the evoked signal is passed to the output of the sense amplifier circuit 360. In at least some embodiments, the risk of saturation of the sense amplifier circuit 360 is increased because of constraints on the sense amplifier circuit 360 or because other components of the system 300 cause the sense amplifier circuit 360 to have relatively limited range and resolution and require that the signals recorded by the processing circuit 302 have relatively high gain. In at least some embodiments, the sense amplifier circuit 360 includes one or more differential amplifiers that use a power supply voltage of 3.3V or less, which further increases the risk of saturation in these embodiments.

In at least some embodiments, saturation of the sense amplifier circuit 360 is prevented by application of a DC offset compensation current from the DC offset compensation circuit 390. In at least some embodiments, the DC offset compensation circuit 390 injects charge into node N1, which is at the input of the sense amplifier circuit 360. There is a capacitance, such as the capacitance of the DC-blocking capacitor C1, at node N1. The current-voltage relationship for a capacitor can be modeled as I=C*(dV/dt). Accordingly, a positive current applied to node with a capacitance causes the voltage at the node to ramp upward, and a negative current applied to the capacitance causes the voltage at the node to ramp downward. When no current is applied to the capacitance, the voltage at the node lowers at a slow rate of speed due to dielectric leakage.

In at least some embodiments, the DC offset compensation circuit 390 is arranged to inject charge at node N1 to cause the DC voltage at the node N1 to be substantially equal to the DC voltage at node N2. In at least some embodiments, sense amplifier circuit 390 is arranged to sense AC signals, and accordingly, it is not useful to sense or amplify the difference in DC voltages between nodes N1 and N2. Further, if the DC voltage difference between nodes N1 and N2 is too great, the sense amplifier circuit 360 will saturate.

Saturation occurs in an amplifier when the output voltage of an amplifier exceeds its range, and therefore the amplifier simply outputs the maximum or minimum voltage instead. Because a saturated amplifier only outputs the minimum or maximum possible voltage, signals that would otherwise be detectable are lost by the saturated amplifier. In some cases, the entire output signal is not amplified to the maximum or minimum voltage, but at least a portion of the signal is, causing the output signal provided by the amplifier to be “clipped.” In these cases, the amplifier is only partially saturated, but still does not provide evoked signal information.

The DC offset compensation current is provided to prevent, or reduce the likelihood of, saturation. In at least some embodiments, the DC offset voltage (between nodes N1 and N2) can be either positive or negative depending on whether the voltage at node N1 or the voltage at node N2 is higher. In at least some embodiments, the DC offset compensation circuit 390 is arranged to determine the DC voltage difference between nodes N1 and N2 and to determine an appropriate current to provide to node N1 to cause the DC voltages at nodes N1 and N2 to be substantially equal to each other.

In at least some embodiments, the current provided by the DC offset compensation circuit 390 is very small relative to the current provided by the stimulation generation circuit 329. In at least some embodiments, the current provided by the DC offset compensation circuit 390 during compensation is no more than 5, 2, or 1 mA or 500, 200, or 100 nA or less. Accordingly, the current provided by the DC offset compensation circuit 390 does not provide any significant contribution to the stimulation of the patient's tissue.

Although FIG. 4 shows the DC offset compensation circuit 390 providing current to node N1, in other embodiments, the DC offset compensation circuit 390 provides current to node N2 instead of or in addition to providing current to node N1. If the DC voltage at node N1 is greater than the DC voltage at node N2, the current provided by the DC offset compensation circuit 390 is positive in embodiments in which the current is provided to node N1. In these embodiments, this positive current will charge the DC-blocking capacitor C1, which will increase the voltage at node N1 and bring the DC voltage offset between nodes N1 and N2 closer to zero. If instead the DC voltage at node N1 is no more than the DC voltage at node N2, in these embodiments, the current provided by the DC offset compensation circuit 390 is negative in examples in which the current is provided to node N1. In these embodiments, this negative current will discharge the DC-blocking capacitor C1, which will decrease the voltage at node N1 and bring the DC voltage offset between nodes N1 and N2 closer to zero.

In at least some embodiments, as the DC offset compensation circuit 390 operates, feedback will eventually cause the DC voltage difference between nodes N1 and N2 to be substantially zero by eventually charging or discharging the DC-blocking capacitor C1 to a level at which the voltage at node N1 is substantially equal to the voltage at node N2. In the embodiment of FIG. 4, the sense amplification input signal SI is a differential input signal. In other embodiments, as discussed above, the sense amplification input signal SI is a single-ended signal. In some embodiments in which the sense amplification input signal SI is a single-ended signal, the sense amplifier circuit 360 is receives a constant reference voltage, and the DC offset compensation circuit 390 provides current to bring the DC voltage at node N1 to be substantially equal to the reference voltage by charging or discharging the DC-blocking capacitor C1 accordingly.

The speed at which the DC offset voltage difference between nodes N1 and N2 is set to zero is strongly influenced by the magnitude of the current provided by the DC offset compensation circuit 390. Causing the DC offset compensation to occur too quickly may be problematic. For example, setting DC offset compensation to occur quickly may affect the frequencies of the AC signals that can be sensed. In general, faster DC offset compensation may prevent the sense amp circuit 310 from sensing lower frequency AC events, because faster compensation will effectively cancel such low-frequency AC events at nodes N1 and N2. In this regard, the DC offset compensation circuit 390 in effect operates as an active high-pass filter, with larger magnitudes of current I1 increasing the cutoff frequency.

Accordingly, using a high magnitude for current I1 may cause potentially interesting low-frequency AC signals to be compensated for, preventing them from being sensed. If it is desired to sense such low-frequency signals in a given application, it may be warranted to adjust the magnitude of current I1 to allow DC offset compensation to occur more slowly. In general, while DC offset compensation is occurring, the signal-to-noise ratio (SNR) of sense amplification output signal SO is reduced. The greater the magnitude of current I1, the lower the SNR of sense amplification output signal SO.

In some embodiments, two different values may be used for I1: a “fast compensation” value and a “slow compensation” value. The “fast compensation” value is larger than the “slow compensation” value. As an example, the “fast compensation” value is several hundred nA (e.g., 500 nA) and the “slow compensation” value is less than 100 nA (e.g., 25 nA). Any other suitable values are used for the “fast compensation” and the “slow compensation.”

Each time the stimulation generation circuit 329 provides a stimulation signal to the tissue (as directed by the processing circuit 302). After the stimulation signal is fully delivered, there is a sensing window in which the evoked response is expected to occur and can be sensed by the sense amplifier circuit 360. For instance, in some embodiments, a sensing window is defined to begin 1, 2, or 5 ms (or any other suitable time period) after the stimulation signal ends. The sensing window can have any suitable duration and can depend on the initiation of the next stimulation signal, the type and position of the tissue that generates the evoked signal, or the like or any combination thereof. In at least some embodiments, the duration of the sensing window is at least 5, 8, 10, 12, 15, or 20 ms. Any other suitable duration can be used.

In at least some embodiments, the processing circuit 302 is arranged to dynamically control the DC offset compensation circuit 390 to provide the DC offset compensation current. In at least some embodiments, the dynamic DC offset compensation provides current I1 at a first magnitude at least during delivery of the stimulation signal to the tissue and, during at least the sensing window, either a) current I1 is provided at a second magnitude, which is substantially smaller than the first magnitude, or b) no current I1 is provided by the DC offset compensation circuit 390. In at least some embodiments, the second magnitude is no more than 33% (one-third), 30%, 25% (one-fourth), 10% (one-tenth), or 5% (one-twentieth) of the first magnitude. Accordingly, the DC offset compensation circuit 390 is dynamically controlled to provide (or not provide) a DC offset compensation current based, at least in part, on the status of the delivery of the stimulation signal.

The greater the magnitude of current I1, the lower the SNR of the sense amplification output signal SO. Because the purpose of the sense amplifier circuit 360 is to sense the evoked response, the sense amplification output signal SO is only of interest during the sense window. To achieve better SNR when the sense amplifier output signal SO is measured, observed, or otherwise sensed, the magnitude of the DC offset compensation current is reduced to the second magnitude or to zero during the sensing window.

Because the sensing window is relatively small, and due to the capacitances present at nodes N1 and N2, it is not expected that the sense amplifier circuit 360 will saturate, either fully or partially, during the sensing window. However, if no DC offset compensation current is provided to node N1 during the sensing windows there is still some chance that the sense amplifier circuit 360 will at least partially saturate. The more current that the DC offset compensation circuit 390 is allowed to provide to node N1, the less the risk of saturation of the sense amplifier circuit 360.

Accordingly, the magnitude of current that the DC offset compensation circuit 390 provides to node N1 is a trade-off between the risk of saturation of the sense amplifier circuit 360 and the SNR of the sense amplification output signal SO. The more current that the DC offset compensation circuit 390 provides to node N1 during the sense window, the lower the SNR of sense amplifier output signal SO, but, also, the lower the risk of saturation of the sense amplifier circuit 360. The choice of current value or no current may depend on a variety of factors.

Accordingly, applying a relatively large DC offset compensation current during delivery of the stimulation signal prevents or reduces the likelihood of saturation of the sense amplifier circuit 360 due to the stimulation signal. Substantially reducing or eliminating the DC offset compensation current during the sensing window increases the SNR of the sense amplification output signal SO to facilitate measuring, observing, or otherwise sensing the evoked signal.

System 300 may have other different configurations other than the specific configurations discussed and shown herein. For example, although the processing circuit 302 is shown as one component in FIG. 4, in some embodiments, the processing circuit 302 is circuitry that includes multiple separate components. For instance, in some embodiments, the processing circuit 302 is circuitry that includes both circuitry for receiving the output of the ADC 311 and entirely separate circuitry for controlling the stimulation generation circuit 329. In at least some embodiments, at least a part of the processing circuit 302 combined with the stimulation generation circuit 329, for example in an IPG or ETS, with another part of the processing circuit 302 being in other components, such as the RC 16 or CP 18.

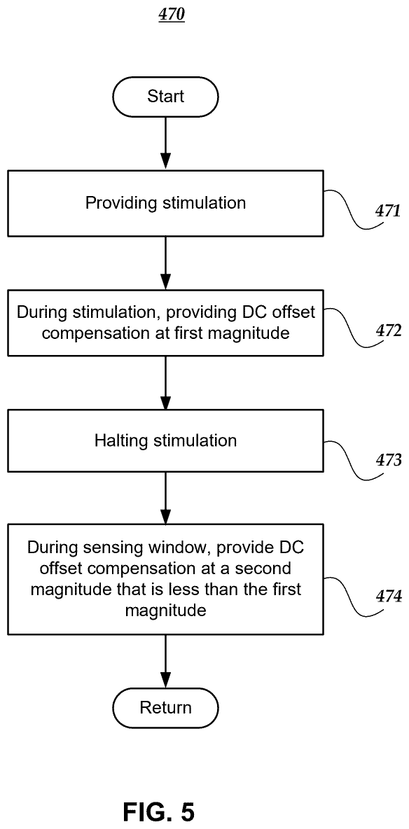

FIG. 5 illustrates an example dataflow for a process (470) for sensing an evoked signal. In at least some embodiments, process 470 may be performed by an embodiment of the processing system 102 of FIG. 2, the processing circuit 302 of FIG. 4, or the like. In at least some embodiments, process 470 proceeds as follows.

At step 471, stimulation is provided using the stimulation generation system or circuit 129/329. At step 472, during the stimulation, DC offset compensation is provided at a first magnitude to prevent or reduce the likelihood of saturation of the sense amplifier circuit.

At step 473, the stimulation is halted. This can be a period between delivery of boluses or pulses of stimulation. At step 474, after halting the stimulation and during a sensing window DC offset compensation is provided at a second magnitude that is less than the first magnitude. The process may then advance to a return block, where other processing is resumed or where the process returns to the start block.

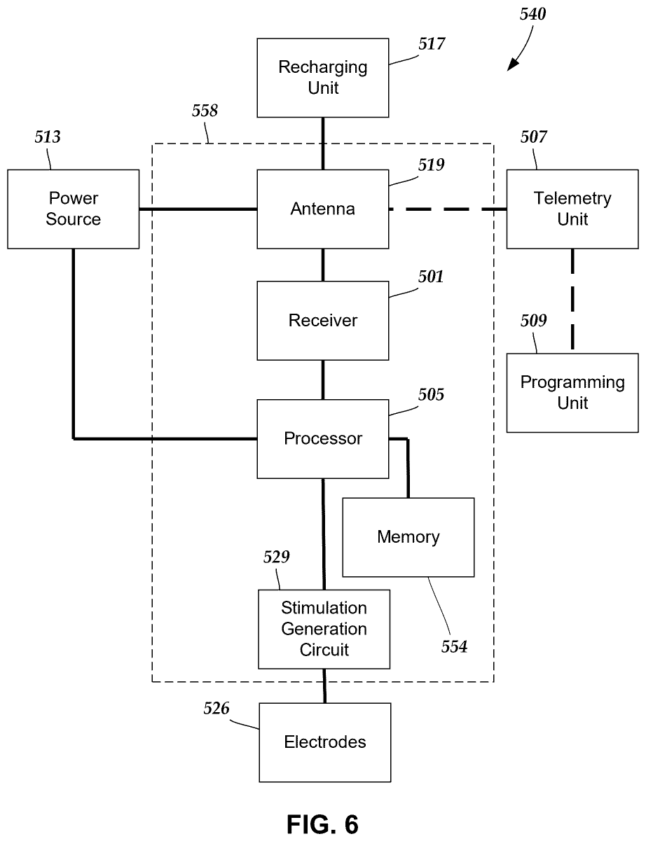

FIG. 6 is a schematic overview of one embodiment of components of an electrical stimulation system 540 including an electronic subassembly 558 disposed within a control module. The electronic subassembly 558 may include one or more components of the IPG (e.g., IPG 14 of FIG. 2). It will be understood that the electrical stimulation system can include more, fewer, or different components and can have a variety of different configurations including those configurations disclosed in the stimulator references cited herein.

Some of the components (for example, a power source 513, an antenna 519, a receiver 501, a processor 505, a memory 554, and a stimulation generation circuit 529) of the electrical stimulation system can be positioned on one or more circuit boards or similar carriers within a sealed housing of an IPG (see e.g., 14 in FIG. 2), if desired. Any suitable power source 513 can be used including, for example, a battery such as a primary battery or a rechargeable battery. Examples of other suitable power sources include super capacitors, nuclear or atomic batteries, mechanical resonators, infrared collectors, thermally powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the optional antenna 519 or a secondary antenna. In at least some embodiments, the antenna 519 (or the secondary antenna) is implemented using the auxiliary electrically-conductive conductor. The external power source can be in a device that is mounted on the skin of the user or in a unit that is provided near the user on a permanent or periodic basis.

If the power source 513 is a rechargeable battery, the battery may be recharged using the optional antenna 519, if desired. Power can be provided to the battery for recharging by inductively coupling the battery through the antenna to a recharging unit 517 external to the user. Examples of such arrangements can be found in the references identified above. The electronic subassembly 558 and, optionally, the power source 513 can be disposed within a control module (e.g., the IPG 14 or the ETS 20 of FIG. 2).

In one embodiment, electrical stimulation signals are generated by the stimulation generation circuit 529 and emitted via the electrodes 26 to stimulate nerve fibers, muscle fibers, or other body tissues near the electrical stimulation system. The processor 505 is generally included to control the timing and electrical characteristics of the electrical stimulation system. For example, the processor 505 can, if desired, control one or more of the timing, frequency, strength, duration, and waveform of the pulses. In addition, the processor 505 can select which electrodes can be used to provide stimulation, if desired. In some embodiments, the processor 505 selects which electrode(s) are cathodes and which electrode(s) are anodes.

In some embodiments, the processor 505 is used to identify which electrodes provide the most useful stimulation of the desired tissue. In at least some embodiments, processor 505 communicates with memory 554. In at least some embodiments, processor 505 executes processor-executable code stored in memory 554. In at least some embodiments, memory 554 is used to store information obtained from sensed evoked signals. Alternatively, instead of or in addition to storing the information obtained from sensed evoked signals in memory 554, the information is output, e.g., by the optional antenna 519.

Any suitable processor can be used and can be as simple as an electronic device that, for example, produces pulses at a regular interval or the processor can be capable of receiving and interpreting instructions from an external programming unit 509 that, for example, allows modification of pulse characteristics. In the illustrated embodiment, the processor 505 is coupled to a receiver 501 which, in turn, is coupled to the optional antenna 519. This allows the processor 505 to receive instructions from an external source to, for example, direct the pulse characteristics and the selection of electrodes, if desired.

Any suitable memory 554 can be used for the processor 552. The memory 554 illustrates a type of computer-readable media, namely computer-readable storage media. Computer-readable storage media may include, but is not limited to, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computer.

In one embodiment, the antenna 519 is capable of receiving signals (e.g., RF signals) from an external telemetry unit 507 which is programmed by the programming unit 509. The programming unit 509 can be external to, or part of, the telemetry unit 507. The telemetry unit 507 can be a device that is worn on the skin of the user or can be carried by the user and can have a form similar to a pager, cellular phone, or remote control, if desired.

As another alternative, the telemetry unit 507 may not be worn or carried by the user but may only be available at a home station or at a clinician's office. The programming unit 509 can be any unit that can provide information to the telemetry unit 507 for transmission to the electrical stimulation system 540. The programming unit 509 can be part of the telemetry unit 507 or can provide signals or information to the telemetry unit 507 via a wireless or wired connection. One example of a suitable programming unit is a computer operated by the user or clinician to send signals to the telemetry unit 507.

The signals sent to the processor 505 via the antenna 519 and the receiver 501 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 540 to cease operation, to start operation, to start charging the battery, or to stop charging the battery. In other embodiments, the stimulation system does not include the antenna 519 or receiver 501 and the processor 505 operates as programmed.

Optionally, the electrical stimulation system 540 may include a transmitter (not shown) coupled to the processor 505 and the antenna 519 for transmitting signals back to the telemetry unit 507 or another unit capable of receiving the signals. For example, the electrical stimulation system 540 may transmit signals indicating whether the electrical stimulation system 540 is operating properly or not or indicating when the battery needs to be charged or the level of charge remaining in the battery. The processor 505 may also be capable of transmitting information about the pulse characteristics so that a user or clinician can determine or verify the characteristics.

The methods and systems described herein may be embodied in many different forms and should not be construed as limited to the embodiments set forth herein. Accordingly, the methods and systems described herein may take the form of an entirely hardware embodiment, an entirely software embodiment or an embodiment combining software and hardware aspects. Systems referenced herein typically include memory and typically include methods for communication with other devices including mobile devices. Methods of communication can include both wired and wireless (for example, RF, optical, or infrared) communications methods and such methods provide another type of computer readable media; namely communication media. Wired communication can include communication over a twisted pair, coaxial cable, fiber optics, wave guides, or the like, or any combination thereof. Wireless communication can include RF, infrared, acoustic, near field communication, Bluetooth™, or the like, or any combination thereof.

It will be understood that each block of the flowcharts, and combinations of blocks in the flowcharts and methods disclosed herein, can be implemented by computer program instructions. These program instructions may be provided to a processor to produce a machine, such that the instructions, which execute on the processor, create ways of implementing the actions specified in the flowchart block or blocks disclosed herein. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computing device. In addition, one or more processes may also be performed concurrently with other processes, or even in a different sequence than illustrated without departing from the scope or spirit of the invention.

The computer program instructions can be stored on any suitable computer-readable medium including, but not limited to, RAM, ROM, EEPROM, flash memory or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

The above specification and examples provide a description of the manufacture and use of the invention. Since many embodiments of the invention can be made without departing from the spirit and scope of the invention, the invention also resides in the claims hereinafter appended.

What is claimed is:

1. A stimulation system, comprising:
a stimulation generation system configured to generate stimulation signals to provide stimulation to tissue of a patient;
an evoked signal sensing system configured to receive an evoked signal from the tissue in response to the stimulation, wherein the evoked signal sensing system comprises
a sense amplifier circuit configured to
receive, at a node, a sense amplification input signal that is based, at least in part, on the received evoked signal, and
provide a sense amplification output signal by amplifying the sense amplification input signal, and
a DC offset compensation circuit configured to provide DC offset compensation at the node; and
a processing system configured to
during the providing of the stimulation, directing the DC offset compensation circuit to provide, at the node, the DC offset compensation at a first magnitude; and
after the providing of the stimulation and during a sensing window for the evoked signal, either a) directing the DC offset compensation circuit to provide, at the node, the DC offset compensation at a second magnitude that is no more than one-third of the first magnitude or b) directing the DC offset compensation circuit to provide, at the node, no DC offset compensation.

2. The stimulation system of claim 1, wherein, during the sensing window, the processing system is configured to direct the DC offset compensation circuit to provide, at the node, the DC offset compensation at the second magnitude that is no more than one-tenth of the first magnitude.

3. The stimulation system of claim 1, wherein, during the sensing window, the processing system is configured to direct the DC offset compensation circuit to provide, at the node, no DC offset compensation.

4. The stimulation system of claim 1, wherein the sense amplifier circuit comprises at least one differential amplifier.

5. The stimulation system of claim 4, further comprising at least one DC-blocking capacitor coupled, or coupleable, to the node.

6. The stimulation system of claim 5, further comprising at least one electrical stimulation lead, the at least one stimulation lead comprising a plurality of electrodes including at least one first electrode and at least one second electrode, wherein the processing system is configured to direct the stimulation generation system to provide the stimulation through the at least first electrode.

7. The stimulation system of claim 6, wherein the sense amplifier circuit is coupled, or coupleable, to the at least one second electrode for receiving the evoked signal from the tissue.

8. The stimulation system of claim 7, wherein, when the sense amplifier circuit is coupled to a one of the at least one second electrode, at least one of the at least one DC-blocking capacitor is coupled between the one of the at least one second electrode and the node.

9. The stimulation system of claim 1, wherein the stimulation system is configured for at least one of deep brain stimulation, spinal cord stimulation, peripheral nerve stimulation, dorsal root ganglion stimulation, or sacral nerve stimulation.

10. The stimulation system of claim 1, further comprising an implantable pulse generator, wherein stimulation generation system and the sense amplifier circuit are part of the implantable pulse generator.

11. The stimulation system of claim 1, further comprising an external stimulator, wherein stimulation generation system and the sense amplifier circuit are part of the external stimulator.

12. A method for sensing an evoked signal using the stimulation system of claim 1, comprising:
providing stimulation to the patient using the stimulation generation system;
during the providing of the stimulation, providing, at the node of the sense amplifier circuit, the DC offset compensation at a first magnitude;
halting the stimulation; and
after the halting of the stimulation and during a sensing window for the evoked signal, either a) providing, at the node, the DC offset compensation at a second magnitude that is no more than one-third of the first magnitude or b) providing, at the node, no DC offset compensation.

13. The method of claim 12, wherein, after the halting of the stimulation and during the sensing window for the evoked signal, the DC offset compensation provided, at the node, is at the second magnitude that is no more than one-tenth of the first magnitude.

14. The method of claim 12, wherein, after the halting of the stimulation and during the sensing window for the evoked signal, no DC offset compensation is provided at the node.

15. The method of claim 12, wherein the stimulation generation system and the sense amplifier circuit are implanted in the patient.

16. An electrical stimulation system, comprising:
at least one electrical stimulation lead comprising a plurality of electrodes; and
a stimulator coupled or coupleable to the at least one electrical stimulation lead, the stimulator comprising
stimulation generation circuitry configured to provide stimulation to tissue of a patient via one or more of the electrodes;
an evoked signal sensing arrangement configured to receive, from at least one of the electrodes, an evoked signal from the tissue in response to the stimulation, wherein the evoked signal sensing arrangement comprises
a sense amplifier circuit configured to
receive, at a node, a sense amplification input signal that is based, at least in part, on the evoked signal received from the at least one of the electrodes, and
provide a sense amplification output signal by amplifying the sense amplification input signal, and
a DC offset compensation circuit configured to provide DC offset compensation at the node, and
a processor configured to
during the providing of the stimulation, directing the DC offset compensation circuit to provide, at the node, the DC offset compensation at a first magnitude; and
after the providing of the stimulation and during a sensing window for the evoked signal, either a) directing the DC offset compensation circuit to provide, at the node, the DC offset compensation at a second magnitude that is no more than one-third of the first magnitude or b) directing the DC offset compensation circuit to provide, at the node, no DC offset compensation.

17. The stimulation system of claim 16, wherein the stimulator is an implantable pulse generator.

18. The stimulation system of claim 16, wherein the stimulator is an external trial stimulator.

19. The stimulation system of claim 16, further comprising at least one DC-blocking capacitor coupled, or coupleable, to the node, wherein at least one of the at least one DC-blocking capacitor is coupled between a one of the electrodes and the node.

20. The stimulation system of claim 19, wherein, when the sense amplifier circuit is coupled to the one of the electrodes, at least one of the at least one DC-blocking capacitor is coupled between the one of the electrodes and the node.
